(19) United States
(12) Patent Application Publication
(10) Pub. No.: US 2025/0381189 A1
(43) Pub. Date: Dec. 18, 2025

(54) PROLONGED RELEASE TOFACITINIB COMPOSITIONS WITHOUT FUNCTIONAL COATING

(71) Applicant: SYNTHON B.V., Nijmegen (NL)

(72) Inventors: Manuel GAGO GUILLAN, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 19/106,475

(22) PCT Filed: Aug. 25, 2023
(86) PCT No.: PCT/EP2023/073382

§ 371 Date: Feb. 25, 2025

(30) Foreign Application Priority Data
Aug. 26, 2022 (EP) 22192473.1

(51) Int. Cl. A61K 31/519; A61K 9/20; A61K 9/28

(52) U.S. Cl. CPC A61K 31/519; A61K 9/2009; A61K 9/2013; A61K 9/2018; A61K 9/2054; A61K 9/2866

(57) ABSTRACT

The present invention relates to a monolithic tablet composition for oral administration of tofacitinib or a pharmaceutically acceptable salt thereof, without a functional coating.

### BACKGROUND OF THE PRESENT INVENTION

Tofacitinib or (3R,4R)-4-methyl-3-(methyl-7H-pyrrolo [2,3-d]pyrimidin-4-ylamino)-β-oxo-1-piperidinepropanenitrile, of the formula:

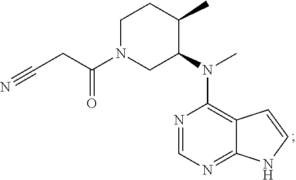 [SMILES: C[C@@H]1CCN(C(=O)CC#N)C[C@@H]1N(C)c1ncnc2[nH]ccc12]

is a reversible inhibitor of the Janus kinase family of kinases (JAK1, JAK2, JAK3 and Tyrosine Kinase 2 (TyK2)). Tofacitinib has been disclosed in WO 2001/042246.

Tofacitinib is indicated for the treatment of adult patients with moderately to severely active rheumatoid arthritis who have had an inadequate response or intolerance to methotrexate. It is marketed as an extended release tablet under the brand name XELJANZ XR® (Pfizer Products Inc.). The tablets are based on osmotic pump technology, wherein the osmotic pressure is used to deliver the tofacitinib at a controlled rate. The tablet insert for XELJANZ XR® tablet describes the tablet as “a pink, oval, extended release film-coated tablet with a drilled hole at one end of the tablet band”.

XELJANZ XR® tablet is a controlled-release formulation, which provides more favourable pharmacokinetic profiles (e.g. reducing the peak variation of drug concentration levels) than the immediate release form, so reducing the side effects and achieving better patient compliance.

XELJANZ XR® drug release profile is very complicated, combining different order kinetics. XELJANZ XR® formulation is described in WO 2014/147526; the formulation is an osmotic pump consisting of a coating made of an insoluble polymer, cellulose acetate, and a core containing tofacitinib citrate, sorbitol, hydroxyethyl cellulose, co-povidone and magnesium stearate. This coating is such that tofacitinib is substantially entirely delivered through the delivery hole, in contrast to delivery via permeation through the coating. The solute concentration gradient, which provides the osmotic force driving the delivery of the drug through the drilled hole, can be maintained constant when solute saturation is present in the tablet core. As the tablet content comes out, solute concentration declines and as well the gradient and the osmotic force driving the drug release.

The typical orifice size in osmotic pumps ranges from about 600 μm to 1 mm. A nominal 600 μm hole usually has a ±100 μm tolerance on diameter, and an allowable ellipticity of 1.0 to 1.5. Although holes of these characteristics and tolerances can be obtained by mechanical means, there is no mechanical method able to work at high manufacturing rates consistent with pharmaceutical manufacturing processes.

In contrast, laser tablet drilling can lead to throughput rates of up to 100,000 tablets/hour having the necessary dimensional tolerances and cosmetic appearance. As a result, laser drilling has become the technology of choice for this type of orifice production.

This technology however requires accepted-rejected system in order to check if the drilled hole on the surface of the tablet meets the specifications. The reject mode is activated as soon as a failed tablet is sensed by the vision system, which causes one or two tablets ahead of the rejected unit to be expelled as well. The reject state only switches off when the system verifies that five tablets in a row meet pass criterion. An additional presence sensor downstream from the blow off verifies that no tablets are passing through the system when the reject condition is set to “on”.

Therefore, the required technology for the manufacturing of the osmotic pump delivery systems is significantly expensive, which is a disadvantage and an economic barrier for many companies.

WO 2012/100949 provides an oral dosage form for modified release comprising tofacitinib and a non-erodible material. In this patent application a monolithic tablet containing a non-erodible material and other components such as pore formers is disclosed. The main disadvantage of this type of delivery systems is the difficulty of the water to penetrate through the material, leading to slow hydration rates. If the centre of the tablet core remains unwetted, this may result in the incomplete dissolution of the drug substance.

WO 2014/174073 discloses a sustained release formulation for oral administration comprising tofacitinib, a hydrophilic polymer and an alkalizing agent. The use of alkalizing agent is proposed for reducing API solubility in acidic pHs, thus obtaining a non-pH dependent release formulation. Alkalizing the tablet core aims to reduce the release of the active ingredient at low pHs where it is more soluble; however, the decrease of the active ingredient solubility by alkalizing the tablet core can limit the drug release at high pHs (for instances at the small intestine) impacting the bioavailability of the drug substance.

WO 2021/038014 discloses a controlled release composition for oral administration comprising tofacitinib and a coating comprising a water-insoluble polymer and a pore former in a specific ratio. This type of delivery systems have many variables affecting the dissolution rate (proportion of pore former, coating weight gain, core tablet composition, the nature of the coating agent . . . ), that result in a less robust formulation. Furthermore, in this application the lag time/control release is achieved using a functional coating. These types of coatings are commonly applied in high weight gains, and they normally need the use of organic solvents for their application.

There is still need of finding an additional oral formulation of tofacitinib which overcomes the problems of the prior art, is advantageously manufactured and is bioequivalent to the commercial tofacitinib tablet XELJANZ XR®.

### DETAILED DESCRIPTION OF THE PRESENT INVENTION

The present invention relates to a monolithic tablet that is advantageously manufactured and provides a bioequivalent formulation of tofacitinib compared with the commercial tablets having an osmotic pump.

As used herein the term “monolithic tablet” refers to a tablet comprising a swellable hydrophilic matrix that delivers the drug in a controlled manner over a long period of time.

A first aspect of the invention relates to a controlled release pharmaceutical tablet with a core comprising tofacitinib or a pharmaceutically acceptable salt thereof and a water soluble pH independent gelling control release polymer with a viscosity grade in a range from 60 to 140 cP in 2% solution in water at 20° C.; wherein the tablet does not have a functional coating.

The dissolution profile provided by the osmotic pump of tofacitinib marketed tablet initially exhibits a short lag time where no drug release takes place. This short lag time corresponds with the diffusion of water through the semi-permeable membrane and the hydration of the tablet core. Afterwards, zero-order kinetic release occurs due to the sustained solute concentration gradient between the tablet core and the dissolution medium. The solute concentration gradient, which provides the osmotic force driving the delivery of the drug through the drilled hole, can be maintained constant whereas solute saturation takes place in the tablet core. As the tablet content comes out, the solute concentration declines and so do the gradient and the osmotic force driving drug release. Ultimately, as a consequence of the decrease of the solute concentration in the tablet core, the dissolution profile shows first-order kinetic release after 3 hours.

Hydrophilic matrix technology has been widely used for oral controlled delivery of various drugs. As well, the combination of barrier membrane and hydrophilic matrix system has been used as a strategy to modulate drug release from hydrophilic matrices and to reduce the overall variability in release. However, it is difficult particularly for very soluble compounds to apply this technology and achieve zero order release. We have surprisingly found that a core comprising tofacitinib or a pharmaceutically acceptable salt thereof and a water soluble pH independent gelling control release polymer with a viscosity grade in a range from 60 to 140 cP in 2% solution in water at 20° C., without a functional coating, results in a zero-order release bioequivalent to XELJANZ XR®.

The monolithic tablet of the current invention is bioequivalent to an osmotic pump system by releasing tofacitinib by diffusion and erosion through the polymeric matrix. Moreover, the technology required for the manufacturing of a monolithic tablet is cheaper and more efficient than the one employed for obtaining osmotic pump systems.

The monolithic tablet of the present invention comprises a core and does not have a functional coating. The core comprises tofacitinib and a water soluble pH independent gelling control release polymer having a viscosity grade in a range from 60 to 140 cP in 2% solution in water at 20° C.

A tablet which does not have a functional coating, means that the core does not have any kind of coating or that the core is covered by a non-functional coating.

The core of the controlled release pharmaceutical tablet of the invention comprises the whole dose of tofacitinib. The word tofacitinib is used herein to refer to tofacitinib free base as well as its pharmaceutically acceptable salts. A preferred salt to be used is the citrate salt.

Tofacitinib free base as well as its pharmaceutically acceptable salts, preferably tofacitinib citrate, is preferably used in an amount of 3% to 15%, more preferably 4% to 12%, most preferably 7% to 10% by weight based on the total core tablet weight.

In the present invention tofacitinib is released from the formulation in a controlled fashion so that after 2 hours, tofacitinib is released in an amount between 40% and 60% and at least 80% of tofacitinib is released after 6 hours in USP III, 20 dpm, 250 ml, SIF pH 6.8, 37° C.

In the present invention, the core of the tablet contains at least one water soluble pH independent gelling control release polymer. The term water soluble pH independent gelling control release polymer means a control release polymer that forms a gel when in contact with water independently of the pH of the water. Such polymers are known in the art and include polyethylene oxide (for example MW:900.000 g/mol; Polyox® 1105 WSR), hydroxypropyl methylcellulose (for example Methocel® K100), hydroxypropyl cellulose, polyvinyl alcohol (for example Parteck® SRP 80), guar gum, carrageenan and combinations thereof. A preferred pH independent gelling control release polymers are soluble polymers such a polyethylene oxide, hydroxypropyl methylcellulose, hydroxypropyl cellulose, polyvinyl alcohol and combinations thereof. More preferably a water soluble pH independent gelling control release polymer are polyethylene oxide and hydroxypropyl methyl cellulose, even more preferably a water soluble pH independent gelling control release polymer is hydroxypropyl methyl cellulose. The amount of the water soluble pH independent gelling control release polymer in the tablet core is preferably in an amount from 20% to 40%, preferably 25% to 40%, more preferably 25% to 37%, even more preferably 30% to 35%, by weight based on the total core tablet weight.

The water soluble pH independent gelling control release polymer in the core of the present invention, has a viscosity grade in a range from 60 to 140 cP in 2% solution in water at 20° C., even more preferably from 70 to 130 cP in 2% solution in water at 20° C., most preferred from 80 to 120 cP in 2% solution in water at 20° C.; measured using a capillary viscosity method as described in USP monograph.

The tablet core may contain additional excipients such as fillers, glidants or lubricants.

Fillers are excipients that are used to increase the bulk volume of a tablet. By combining a filler with the active pharmaceutical ingredient, the final product is given adequate weight and size to assist in production and handling.

The tablet core of the present invention contains preferably at least one filler.

Fillers are preferably used in an amount of from 30% to 80%, more preferably 40% to 70%, most preferably 45% to 60% by weight based on the total core tablet weight. Suitable examples of fillers to be used in accordance with the present invention include mannitol, sorbitol, microcrystalline cellulose, lactose, phosphates, starch, pregelatinized starch, and combinations thereof.

In a preferred embodiment of the present invention, the fillers to be used are microcrystalline cellulose, lactose or mixtures thereof. In a further preferred embodiment of the present invention, the fillers to be used are microcrystalline cellulose and lactose.

The tablet core may also contain glidants and/or lubricants.

Glidants enhance product flow by reducing interparticulate friction. A suitable example is colloidal silicon dioxide. Glidants are preferably used in a total amount of from 0.05% to 5%, more preferably 0.1% to 2%, most preferably 0.2% to 1.0% by weight based on the total core tablet weight.

Lubricants are generally used in order to reduce sliding friction. In particular, to decrease friction at the interface between a tablet's surface and the die wall during ejection, and reduce wear on punches and dies. Suitable lubricants to be used in accordance with the present invention include magnesium stearate, calcium stearate, stearic acid, glyceryl behenate, hydrogenated vegetable oil, and sodium stearyl fumarate. Lubricants are preferably used in a total amount of from 0.05% to 5%, more preferably 0.5% to 3%, most preferably 0.8% to 2.5% by weight based on the total core tablet weight. A preferred lubricant is magnesium stearate.

In the present invention, the tablet core is uncoated or coated with a coating, which is a non-functional coating.

A coating is defined as a film, which is a thin membrane, applied onto the surface of tablets, capsules, granules and spheroidal unit dosage forms.

A non-functional coating is designed to dissolve rapidly in the gastrointestinal tract without any significant impact on the rate of drug release in the gut; it is used for esthetical reasons. The monolithic tablet of the present invention, has achieved a bioequivalent formulation to XELJANZ XR® without using any of the more complex functional coatings disclosed in the prior art.

Examples of non-functional coatings are one or more species selected from among low viscosity grade hydroxypropyl methylcellulose (hypromellose), ethyl cellulose, low viscosity grade hydroxypropyl cellulose, polyvinyl alcohol, povidone, sucrose, and mannitol; a most preferred coatings are low viscosity grade hypromellose, low viscosity grade hydroxypropyl cellulose and polyvinyl alcohol; even more preferred is low viscosity grade hypromellose.

The coating of the current invention is in an amount of 1% to 5%, even more preferably 2 to 4% w/w of weight gain in relation to core tablet mass.

The coating may be prepared using conventional methods well-known in the art. The coating is applied by spraying a suspension of the coating components over the tablet. Such suspension is prepared by dispersing the coating components in a suitable solvent. Suitable solvents are purified water, ethanol, isopropyl alcohol, methylene chloride or mixtures thereof. Preferable suitable solvent is water.

Optionally other excipients like plasticizer (e.g. polyethylene glycol, triacetin and triethyl citrate), colourants (e.g. iron oxides, titanium dioxide) etc. are added to obtain a homogeneous suspension. Optionally a ready to use mix comprising all coating components may be used. The obtained suspension is sprayed over the tablets.

In a preferred embodiment, the tablet comprises:
1. A core comprising:
   a. Tofacitinib or a pharmaceutically acceptable salt in an amount of from 3% to 15% w/w by weight based on the total core tablet weight;
   b. Hydroxypropyl methylcellulose with a viscosity grade in a range from 60 to 140 cP s in 2% solution in water at 20° C. in an amount of from 20 to 40% w/w by weight based on the total core tablet weight;
   c. One or more filler in an amount of from 40% to 70% w/w by weight based on the total core tablet weight;
   d. One or more glidant in an amount of from 0.1% to 2.0% w/w by weight based on the total core tablet weight;
   e. One or more lubricant in an amount of from 0.5% to 3% w/w by weight based on the total core tablet weight;
2. A non-functional coating in an amount from 2% to 4% w/w of weight gain in relation to core tablet mass.
   The tablet of the invention can be made using conventional methods and equipment well-known in the art for example direct compression, wet granulation or dry granulation. In a preferred embodiment the tablet of the invention is prepared by direct compression.

The tablet composition in accordance with the present invention is bioequivalent in vivo to the commercially available tofacitinib citrate tablets XELJANZ XR®.

The present invention is illustrated by the following Examples. Tables 1 and 2 provide compositions of the tablets prepared in examples 1 and 2 accordingly.

### Example 1: Prolonged Release Formulation Containing 40% w/w of Methocel K100 LV (HPMC 100 cP in 2% Solution at 20° C.) in the Tablet Core

355.3 grams of tofacitinib citrate, 1600.0 grams of Methocel K100 LV CR and 20.0 grams of colloidal anhydrous silica were weighed and mixed. 972.3 grams of microcrystalline cellulose and 972.3 grams of lactose monohydrate were weighed, and added together with the previous blend (1); the components were mixed. 80.0 grams of magnesium stearate was mixed to the previous blend (2); This blend (3) was then compressed in a rotary tabletting machine.

Prepared tablet cores were added to the coater pan. A ready to use coating based on low viscosity grade hypromellose was added into purified water and mixed. Then the suspension was sprayed over the tablets previously heated in the coating pan, until the tablets achieved a 3% weight increase.

TABLE 1

| | Formulation | |
|---|---|---|
| Ingredients | Weight/tab (mg) | % |
| Tofacitinib citrate | 17.766 | 8.883 |
| Hydroxypropyl methylcellulose | 80.000 | 40.000 |
| Microcrystalline cellulose | 48.617 | 24.309 |
| Lactose monohydrate | 48.617 | 24.309 |
| Colloidal anhydrous silica | 1.000 | 0.500 |
| Magnesium stearate | 4.000 | 2.000 |
| Ready to use coating based on hypromellose | 6.000 | 3.000 |
| (low viscosity grade) | | |

### Example 2: Prolonged Release Formulation Containing 34% w/w of Methocel K100 LV (HPMC 100 cP in 2% Solution at 20° C.) in the Tablet Core

710.6 grams of tofacitinib citrate, 2720.0 grams of Methocel K100 LV CR and 40.0 grams of colloidal anhydrous silica were weighed and mixed. 2184.7 grams of microcrystalline cellulose and 2184.7 grams of lactose monohydrate were weighed, and added together with the previous blend (1); the components were mixed. 160.0 grams of magnesium stearate was mixed to the previous blend (2); This blend (3) was then compressed in a rotary tabletting machine.

Prepared tablet cores were added to the coater pan. A ready to use coating based on low viscosity grade hypromellose was added into purified water and mixed. Then the suspension was sprayed over the tablets previously heated in the coating pan, until the tablets achieved a 3% weight increase.

TABLE 2

| | Formulation | |
|---|---|---|
| Ingredients | Weight/tab (mg) | % |
| Tofacitinib citrate | 17.766 | 8.883 |
| Hydroxypropyl methylcellulose | 68.000 | 34.000 |
| Microcrystalline cellulose | 54.617 | 27.309 |
| Lactose monohydrate | 54.617 | 27.309 |
| Colloidal anhydrous silica | 1.000 | 0.500 |
| Magnesium stearate | 4.000 | 2.000 |
| Ready to use coating based on low viscosity | 6.000 | 3.000 |
| grade hypromellose | | |

1. A controlled release pharmaceutical tablet with a core comprising tofacitinib or a pharmaceutically acceptable salt thereof and a water soluble pH independent gelling control release polymer with a viscosity grade in a range from 60 to 140 cP in 2% solution in water at 20° C.; wherein the tablet does not have a functional coating.

2. The tablet according to claim 1 such that tofacitinib is released from the tablet, in a controlled fashion so that, after 2 hours, tofacitinib is released in an amount between 40% and 60% and at least 80% of tofacitinib is released after 6 hours in USP III, 20 dpm, 250 ml, SIF pH 6.8, 37° C.

3. The tablet according to claim 1, wherein tofacitinib is present in an amount of from 3% to 15% by weight based on the total tablet core weight.

4. The tablet according to claim 1, wherein said water soluble pH independent gelling control release polymer in said core is in an amount from 20 to 40% by weight to the total tablet core weight.

5. The tablet according to claim 1, wherein said water soluble pH independent gelling control release polymer in said core is selected from the group consisting of polyethylene oxide, hydroxypropyl methylcellulose, hydroxypropyl cellulose, polyvinyl alcohol, and combinations thereof.

6. The tablet according to claim 5, wherein said water soluble pH independent gelling control release polymer in said core is hydroxypropyl methylcellulose.

7. The tablet according to claim 1, which has a non-functional coating.

8. The tablet according to claim 7 wherein the non-functional coating is in an amount 1% to 5% w/w in relation to the core tablet weight.

9. The tablet according to claim 7, wherein said non-functional coating is selected from the group consisting low viscosity grade hypromellose, low viscosity grade hydroxypropyl cellulose and polyvinyl alcohol and combinations thereof, preferably low viscosity grade hypromellose.

10. The tablet according to claim 1, wherein said core further comprises one or more excipients selected from the group of filler, glidant and lubricant.

11. The tablet according to claim 1, wherein said core comprises:
a) tofacitinib or a pharmaceutically acceptable salt in an amount of from 3% to 15% w/w by weight based on the total core tablet weight;
b) hydroxypropyl methylcellulose with a viscosity grade in a range from 60 to 140 cP in 2% solution in water at 20° C. in an amount from 20 to 40% w/w by weight based on the total core tablet weight;
c) one or more fillers in an amount of from 40% to 70% w/w by weight based on the total core tablet weight;
d) one or more glidants in an amount of from 0.1% to 2.0% w/w by weight based on the total core tablet weight; and
e) one or more lubricants in an amount of from 0.5% to 3% w/w by weight based on the total core tablet weight.

12. The tablet according to claim 1, wherein the water soluble pH independent gelling control release polymer in the core has a viscosity grade in a range from 80 to 120 cP in 2% solution in water at 20° C.

13. The tablet according to claim 1, wherein said filler is selected from the group comprising mannitol, sorbitol, microcrystalline cellulose, lactose, phosphates, starch and combinations thereof.

14. The tablet according to claim 1, wherein said filler is a combination of microcrystalline cellulose and lactose.

15. The tablet according to claim 1, wherein tofacitinib is in the form of tofacitinib citrate.
